(19) United States
(12) Patent Application Publication
(10) Pub. No.: US 2024/0047078 A1
(43) Pub. Date: Feb. 8, 2024

(54) ENFORCING SOCIAL DISTANCING USING TECHNOLOGY

(71) Applicant: Wells Fargo Bank, N.A., San Francisco, CA (US)

(72) Inventor: Brian J. Jacobsen, Elm Grove, WI (US)

(21) Appl. No.: 17/247,104

(22) Filed: Nov. 30, 2020

(51) Int. Cl. G16H 50/80

(52) U.S. Cl. CPC G16H 50/80

(57) ABSTRACT

Various examples are directed to computer-implemented systems and methods for enforcing social distancing using technology. A method includes communicating with one or more sensors to determine a location of each of a plurality of users within a premises, and determining whether a first user is within a predetermined distance from at least a second user. An alert is sent to a user device of the first user if the first user is within the predetermined distance from at least the second user. A datastore is queried to determine whether the premises is affiliated with an entity that offers rewards for maintaining social distancing. A user profile of the first user is updated to indicate a reward is available to the first user if the computer system determines that the first user does not enter the predetermined distance from at least the second user for a predetermined duration of time.

### CROSS REFERENCE TO RELATED APPLICATIONS

This application is related to co-pending, commonly assigned U.S. patent application Ser. No. ______, entitled “SYSTEM AND METHOD FOR SENSOR-BASED MASK DETECTION” (Attorney Docket No. 4423.376US1), and related to co-pending, commonly assigned U.S. patent application Ser. No. ______, entitled “SYSTEM AND METHOD FOR SENSOR-BASED SOCIAL DISTANCING” (Attorney Docket No. 4423.378US1), both filed on even date herewith, which are hereby incorporated by reference herein in their entirety.

### BACKGROUND

In view of the proliferation of contagious airborne viruses, maintaining distance from other people in public, commonly referred to as “social distancing,” has significant importance. Some experts have opined that social distancing is one of the most effective tools in limiting the spread of Covid-19 and other communicable airborne viruses. Some people find it difficult to remember to socially distance from others when in a retail setting or other public enclosed space.

### BRIEF DESCRIPTION OF THE DRAWINGS

In the drawings, which are not necessarily drawn to scale, like numerals may describe similar components in different views. Like numerals having different letter suffixes may represent different instances of similar components. Some embodiments are illustrated by way of example, and not of limitation, in the figures of the accompanying drawings, in which:

FIG. 1 illustrates an example embodiment of a computer system for use in the present subject matter;

FIG. 2 illustrates an exemplary infrastructure for use in the present subject matter;

FIG. 3A illustrates an example embodiment of a method for assisting in social distancing compliance, according to various embodiments;

FIG. 3B illustrates an example embodiment of a method for enforcing social distancing using technology, according to various embodiments;

FIG. 4 illustrates example embodiments of sensors for use at a premises, according to various embodiments; and

FIG. 5 is a block diagram of a machine in the example form of a computer system within which a set of instructions may be executed, for causing the machine to perform any one or more of the methodologies discussed herein.

### DETAILED DESCRIPTION

A person may not be aware of other persons in their vicinity when in public spaces. Therefore, there is a technical problem to ensure that people maintain a set minimum distance spacing between them for proper social distancing. The present subject matter provides systems and methods to enforce social distancing using technology. In one embodiment, the present subject matter is included in an application on a person's smartphone. If a person or user is closer to another person than a predetermined proximity limit (e.g., a programmable and adjustable distance or proximity limit), the user may receive an alert notifying them they are too close to the other person, and directing them to move away to obtain the proper social distance, in various embodiments. A variety of location and positioning methods can be used to measure the proximity between individuals without departing from the scope of the present subject matter.

In various embodiments, the present subject matter provides incentives for a person to avoid close proximity to another person, such as using rewards and alerts for participation and compliance with a social distancing program and with actual social distancing, A reward system may have one component or two components, in various embodiments, a first component rewards the user for participation or “signing up” or downloading the application to be part of the social distancing application. A second component rewards the user for complying with social distancing, in various embodiments. For example, a user that completes a shopping trip to a participating retail location while maintaining social distancing may receive an additional discount on top of the participation discount. In some examples, the user may receive an additional discount for responding to an alert by increasing the distance from other persons, thus complying with social distancing.

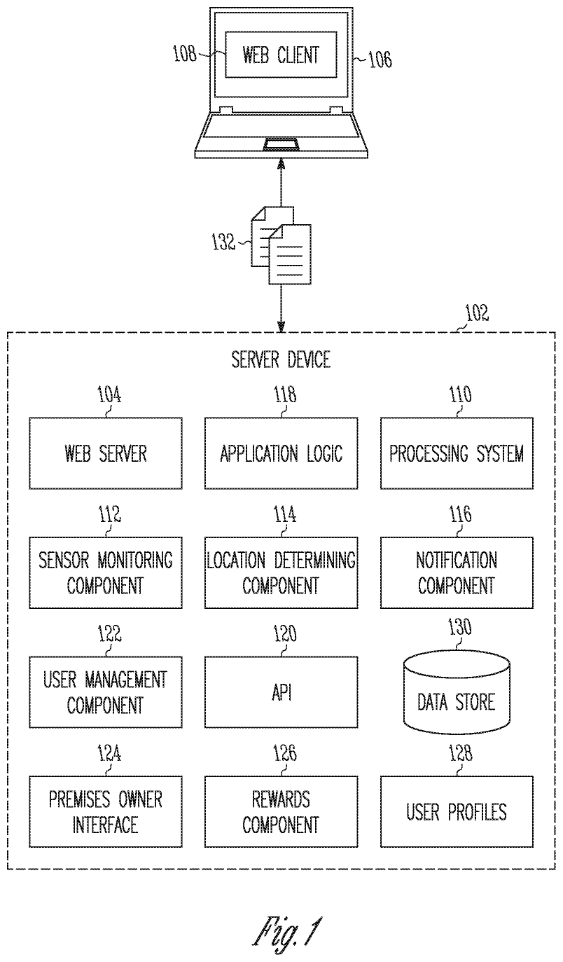

FIG. 1 illustrates an example embodiment of a computer system for use in the present subject matter. FIG. 1 comprises a server device 102, web server 104, client device 106, web client 108, processing system 110, sensor monitoring component 112, location determining component 114, notification component 116, application logic 118, application programming interface (API) 120, user management component 122, premises owner interface 124, rewards component 126, user profiles 128, data store 130, and data 132.

Client device 106 may be a computing device which may be, but is not limited to, a smartphone, tablet, laptop, multi-processor system, microprocessor-based or programmable consumer electronics, game console, set-top box, or other device that a user utilizes to communicate over a network. In various examples, a computing device includes a display module (not shown) to display information (e.g., in the form of specially configured user interfaces). In some embodiments, computing devices may comprise one or more of a touch screen, camera, keyboard, microphone, or Global Positioning System (GPS) device. Client device 106 may be associated with one or more entities that interact with server device 102. An entity may be an individual, group of individuals, or company in various examples.

Client device 106 and Server device 102 may communicate via a network (not shown). The network may include local-area networks (LAN), wide-area networks (WAN), wireless networks (e.g., 802.11 or cellular network), the Public Switched Telephone Network (PSTN) Network, ad hoc networks, cellular, personal area networks or peer-to-peer (e.g., Bluetooth®, Wi-Fi Direct), or other combinations or permutations of network protocols and network types. The network may include a single Local Area Network (LAN) or Wide-Area. Network (WAN), or combinations of LAN's or WAN's, such as the Internet.

Client device 106 and server device 102 may communicate data 132 over the network. Data 132 may be, but is not limited to, search requests, search results, market offers (as discussed in more detail below), user information, validation results, and decision results.

In some examples, the communication may occur using an application programming interface (API) such as API 120. An API provides a method for computing processes to exchange data. A web-based API (e.g., API 120) may permit communications between two or more computing devices such as a client and a server.

Server device 102 is illustrated as set of separate elements (e.g., component, logic, etc.). However, the functionality of individual elements may be performed by a single element. An element may represent computer program code that is executable by processing system 110. The program code may be stored on a storage device (e.g., data store 130) and loaded into a memory of the processing system 110 for execution. Portions of the program code may be executed in a parallel across multiple processing units (e.g., a core of a general-purpose computer processor, a graphical processing unit, an application specific integrated circuit, etc.) of processing system 110. Execution of the code may be performed on a single device or distributed across multiple devices. In some example, the program code may be executed on a cloud platform (e.g., MICROSOFT AZURE® and AMAZON EC2®) using shared computing infrastructure.

Server device 102 may include web server 104 to enable data exchanges with client device 106 via web client 108. Although generally discussed in the context of delivering webpages via the Hypertext Transfer Protocol (HTTP), other network protocols may be utilized by web server 104 (e.g., File Transfer Protocol, Telnet, Secure Shell, etc.). A user may enter in a uniform resource identifier (URI) into web client 108 (e.g., the INTERNET EXPLORER® web browser by Microsoft Corporation or SAFARI® web browser by Apple Inc.) that corresponds to the logical location (e.g., an Internet Protocol address) of web server 104. In response, web server 104 may transmit a web page that is rendered on a display device of a client device (e.g., a mobile phone, desktop computer, etc.).

Additionally, web server 104 may enable a user to interact with one or more web applications provided in a transmitted web page. A web application may provide user interface (UI) components that are rendered on a display device of client device 106. The user may interact (e.g., select, move, enter text into) with the UI components, and based on the interaction, the web application may update one or more portions of the web page. A web application may be executed in whole, or in part, locally on client device 106. The web application may populate the UI components with data from external sources or internal sources (e.g., data store 130) in various examples.

For example, server device 102 may provide a web application (e.g., user management component 122) to a user to provide an interface for the user to interact with the application. Data store 130 may store user information and/or rewards information, including unique identifiers to identify users. That is, in the various examples described herein, an application may provide by the server 102 that permits a user (e.g., via the client device 106) to receive, view, access, and redeem rewards. As further described herein, rewards may be offered for activity related to social distancing. Further, the application provided by the server 102 may provide one or more alerts. For instance, alerts may also be related to social distancing. In certain examples, the application may also facilitate communication with one more sensors at or near a user device. For instance, this may be facilitated using integrated technology of the client device 106, such as wireless communication technology of the user device (e.g., a smartphone).

The web application may be executed according to application logic 118. Application logic 118 may use the various elements of server device 102 to implement the web application. For example, application logic 118 may issue API calls to retrieve or store data from data store 130 and transmit it for display on client device 106. Similarly, data entered by a user into a UI component may be transmitted using API 120 back to the web server 104.

Data store 130 is depicted as singular element but may in actuality be multiple data stores. The specific storage layout and model used in by data store 130 may take a number of forms—indeed, a data store 130 may utilize multiple models. Data store 130 may be, but is not limited to, a relational database (e.g., SQL), non-relational database (NoSQL) a flat file database, object model, document details model, graph database, shared ledger (e.g., blockchain), or a file system hierarchy. Data store 130 may store data on one or more storage devices (e.g., a hard disk, random access memory (RAM), etc.). The storage devices may be in standalone arrays, part of one or more servers, and may be located in one or more geographic areas.

User profiles 128 may store the profiles of users that interact with server device 102. A user profile may include a user identifier (e.g., a username) and a password. A user profile may also include a user's name, address, phone number, account numbers, unique identifiers, and other information.

In various embodiments, the sensor monitoring component 112 can assist with determining location and proximity of one or more users or persons, by communicating with a sensor or sensors carried with the users or located at the premises. The location determining component 114 can use received signals from the sensor monitoring component 112 and/or geolocation data from user devices to determine a location of each of a plurality of users within a specific geographic location (e.g., business premises) corresponding to a retail establishment, or other entity, and to determine whether a user of the plurality of users is within a predetermined distance from other users of the plurality of users, in various embodiments. A number of different sensors or signals can be used, including but not limited to global positioning system (GPS), Bluetooth, Bluetooth Low Energy (BLE), near field communication (NFC), radio frequency identification (RFID) and Wi-Fi. The system can detect range between individuals using device communications and/or signal strength of one or more of the sensors or signals, in various embodiments.

According to various embodiments, the notification component 116 can send an alert to a user device if the user is within the predetermined distance from any of the other users. The rewards component 126 can determine whether the specific geographic location is affiliated with an entity that offers rewards to users for maintaining social distancing, and/or provide rewards to the user when the user has not received the alert for a predetermined duration at the premises that offers rewards to users for maintaining social distancing, in various embodiments. The present subject matter may further provide internal tools, such as a look-up tool or premises owner interface 124, which may enable premises owner employees to review and update available rewards offers and statuses.

For instance, an application may be provided by the server 102 that permits a retail user (e.g., via the client device 106) to offer and update available rewards. That is, the application may provide one or more user interfaces that permit a retail user to define a rewards offer, and the conditions associated with that rewards offer. Satisfaction of the conditions may result in the reward being delivered. For example, the conditions associated with a rewards offer may be associated with a potential customer maintaining social distancing when a location of that potential customer corresponds to a geographic location of a retail establishment of the retail user.

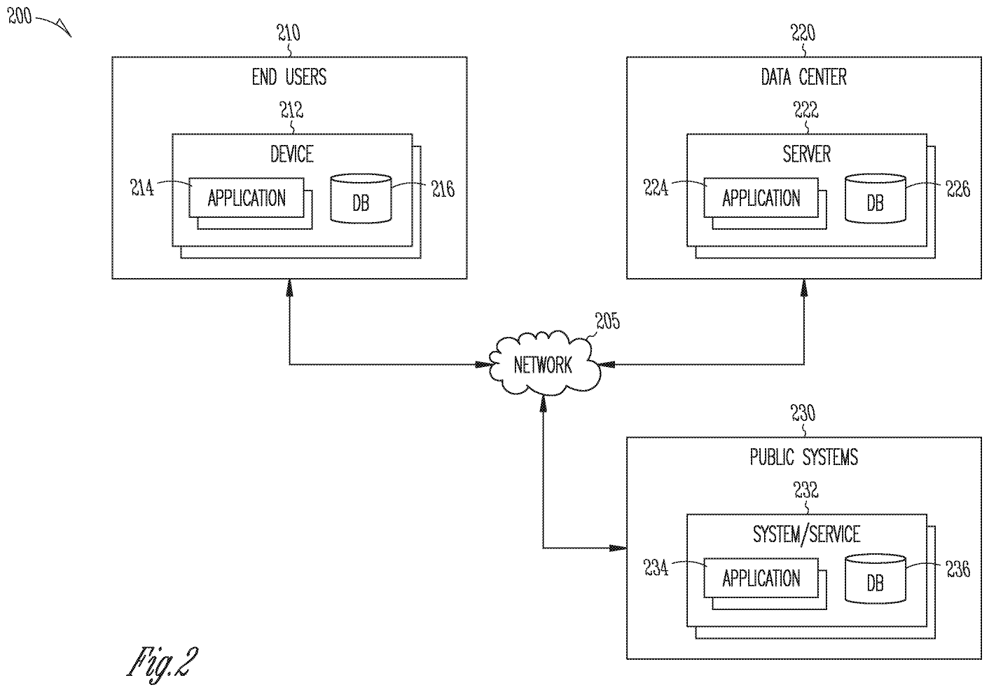

FIG. 2 illustrates an exemplary infrastructure for providing the system of the present subject matter. The infrastructure may comprise a distributed system 200 that may include a client-server architecture or cloud computing system. Distributed system 200 may have one or more end users 210. An end user 210 may have various computing devices 212, which may be a machine 500 as described below. The end-user computing devices 212 may comprise applications 214 that are either designed to execute in a stand-alone manner, or interact with other applications 214 located on the device 212 or accessible via the network 205. These devices 212 may also comprise a data store 216 that holds data locally, the data being potentially accessible by the local applications 214 or by remote applications.

The system 200 may also include one or more data centers 220. A data center 220 may be a server 222 or the like associated with a business entity that an end user 210 may interact with. The business entity may be a computer service provider, as may be the case for a cloud services provider, or it may be a consumer product or service provider, such as a retailer. The data center 220 may comprise one or more applications 224 and databases 226 that are designed to interface with the applications 214 and databases 216 of end-user devices 212. Data centers 220 may represent facilities in different geographic locations where the servers 222 may be located. Each of the servers 222 may be in the form of a machine(s) 500.

The system 200 may also include publicly available systems 230 that comprise various systems or services 232, including applications 234 and their respective databases 236. Such applications 234 may include news and other information feeds, search engines, social media applications, and the like. The systems or services 232 may be provided as comprising a machine(s) 500.

The end-user devices 212, data center servers 222, and public systems or services 232 may be configured to connect with each other via the network 205, and access to the network by machines may be made via a common connection point or different connection points, e.g. a wireless connection point and a wired connection. Any combination of common or different connections points may be present, and any combination of wired and wireless connection points may be present as well. The network 205, end users 210, data centers 220, and public systems 230 may include network hardware such as routers, switches, load balancers and/or other network devices.

Other implementations of the system 200 are also possible. For example, devices other than the client devices 212 and servers 222 shown may be included in the system 200. In an implementation, one or more additional servers may operate as a cloud infrastructure control, from which servers and/or clients of the cloud infrastructure are monitored, controlled and/or configured. For example, some or all of the techniques described herein may operate on these cloud infrastructure control servers. Alternatively, or in addition, some or all of the techniques described herein may operate on the servers 222.

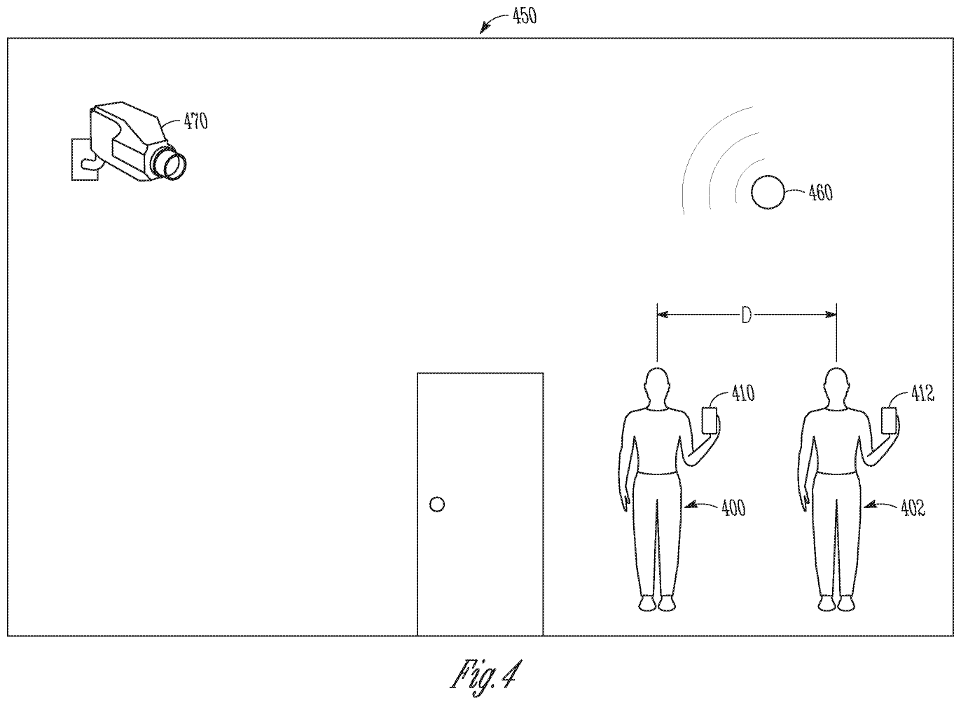

The present subject matter provides incentives for a person to avoid coming too close to another person. In some embodiments (such as shown in FIG. 4), a user device (e.g., a smartphone or wearable device) of a user will detect a user device (or other wireless, such as Bluetooth, activated device) of another person in close proximity using sensor monitoring component 112 and/or location determining component 114, and provide a notification or warning if the distance between the user device and the device of the other person is less than an adjustable threshold using notification component 116. In various embodiments, a smartphone can use a personal hotspot WiFi to determine proximity of the other person's device. In some embodiments, proximity (e.g., predetermined distance) can be limited to six feet. In other embodiments, a proximity limit between a device of a first person and a device of a second person can be set to 8 feet, to account for where on the other person the device is located, to keep people six feet apart. Other predetermined proximity limit values (distances) can be used without departing from the scope of the present subject matter.

If devices are closer than the predetermined proximity limit, the person may receive an alert on a user device, such as a smartphone or wearable device, notifying them they are too close to another person and directing them to move away to obtain the proper social distance, using notification component 116. The alert can be similar to an automobile notifying you that you are too close to a curb or another vehicle, for example. In various embodiments, if a person is using a wearable device such as Google Glass, the camera can be used for proximity location and approximation.

The present subject matter is configured for single or multiple person usage. For example, if only one person in the premises has the present application running on their device, the application will locate other people and enforce social distancing from the other people, but only provide alerts and rewards to the one person having the present application running on their device. In some embodiments, the proximity detection is done from peer-to-peer using Bluetooth, WiFi, or other communication protocols between user devices in communication with sensor monitoring component 112. In some embodiments, the premises owner (such as a retailer or financial institution) can use its own network, such as an existing WiFi network having beacons or repeaters, to locate individuals for purposes of the present subject matter using location determining component 114.

While in some examples, the sensor or sensors may communicate via discrete signals, in various other examples, a continuous stream of communication may be established between the sensor and the system. Accordingly, in some examples, the person may be periodically “checked” to ascertain if the person is socially distancing, or if the person is consistently social distancing over a given period of time. The period of time (e.g., frequency of polling) may be one minute, five minutes, or thirty minutes, to name a few examples. While described herein as a signal to the sensor from the system, and a response from the sensor to the system, in various other examples a signal may be sent from the sensor to the system. That is, the sensor may broadcast a beacon, the detection of which (e.g., by the smartphone) may confirm that the person is socially distancing.

In some embodiments, the premises owner may have a surveillance system with cameras that is used to locate individuals for purposes of the present subject matter. The surveillance system may be capable of tagging a person to identify them as they enter the premises, such as by using a motion sensor, camera (if within line of sight) or infrared sensor (triggered by body heat threshold) to detect the person. If the detected person has the application of the present subject matter running on their user device, or included in an application accessed through a web browser using the person's user device, then identification can be used with the detection (or tagging) of the person and they can be followed while on the premises to enforce social distancing, in various embodiments. If the person does not have the application running, they can still be monitored with respect to other individuals for proximity and social distancing, but will not receive the rewards or alerts for participation and compliance.

In some embodiments, a premises owner such as a retailer may use their retail software application to enable tracking of customers to enforce social distancing, as a type of location determining component 114. The retailer may provide an “opt-in” on the application to allow the application to track the user while on the retail premises, in an embodiment. The application can then use the geotracking of the customer/user device to provide social distancing tracking. In one embodiment, security tags (such as those used on expensive items in a retail store to prevent shoplifting) can be used to track individuals. A membership card of a user for a retailer (such as a rewards card) can have a unique identifier, include an RFID chip, or be made of a material similar to a security tag to enable tracking within the store, in some embodiments. The social distancing application of the present subject matter can be a cloud-based system managed by the retailer, or by a third party, in various embodiments. If a person does not have the store membership card, they can still be monitored with respect to other individuals for proximity and social distancing, but will not receive the rewards or alerts for participation and compliance.

While described in some examples as including a single sensor, in various other embodiments may include a plurality of sensors. Each sensor of the plurality may be the same type of sensor, or a different type of sensor. For example, the system may communicate with a first sensor of a first type, and a second sensor of second type. A combination of different sensor types offers the benefit of improved data reporting as it offers a more comprehensive report of collected information.

As previously discussed herein, in various examples the sensor or sensors may be continuously or dynamically polled to determine if the user is social distancing. In certain other examples, the sensor or sensors may continuously or periodically transmit a signal (e.g., a beacon) when the sensor or sensors are sensing information. Such a beacon may be located and received by the person's smartphone, A continuous or dynamic communication stream with the sensors ensures that the person is consistently social distancing.

The present subject matter provides for rewards and alerts for participation and compliance with the social distancing program and with actual social distancing, using rewards component 126. For example, a person that uses the application of the present subject matter can get alerts sent to their device in real time for getting “too close” to another person using notification component 116, based on the social distancing policy set by the application or set by the premises owner (based on local rules, regulations or retail requirements). The premises owner may refer to lessees or other entities that controls a location for purposes of the person's visit to the premises, it is not necessary for the premises owner to legally own the premises for the purposes of this application. The alerts can include vibration or audible alerts such as chimes or spoken warnings to move away from other people to comply with social distancing.

In addition, the subject matter of the present application may provide for a reward system (such as by using rewards component 126). The reward system may have two portions, in various embodiments, a first portion rewards the individual for participation or “signing up” or downloading the application to be part of the social distancing application. For example, a person may be granted a 5% discount on retail purchases for signing up to use the application of the present subject matter.

The second portion rewards the individual for complying with social distancing. For example, a person that completes a shopping trip to the retail location while maintaining social distancing may receive an additional discount on top of the participation discount. Likewise, the person may receive an additional discount for responding to an alert by increasing the distance from other persons, thus complying with social distancing. For example, if a person gets an alert (a notification to move away) and responds by moving away, they could get an additional reward or incentive of an additional 1% off of their purchases in the store that day. In another example, if a person navigates their entire shopping trip without violating social distancing and thus not getting any alerts, they may receive an additional 2% off instore or future purchases. Thus, the application of the present subject matter encourages people to shop during off-peak hours to get a discount. For example, a person using this application may choose to shop at 2 pm to avoid the lunch rush, to increase their odds of getting a reward, thus enhancing social distancing and reducing possibility of transmission of communicable diseases. In this manner, the present subject matter provides incentives to people to socially distance and benefit their own health as well as others, whether or not they share the same premises at the same time.

In some embodiments, each retail location can customize the distance rules, such as by using premises owner interface 124. For example, in some stores people in adjacent aisles may be closer than six feet apart, but deemed not in violation of social distancing because of large dividers or high shelves between the aisles. In various embodiments, the application of the present subject matter knows details of the store layout, or uses available video to determine store features that automatically and/or dynamically mitigate distancing.

The application can provide other positive or negative incentives for social distancing compliance, in various embodiments. For example, a notification can be provided on a user device if not in compliance, using notification component 116. Likewise, a social media post to user-selected accountability partners (similar to a dieting regime) can be made to let others know if the user is in compliance. This can provide peer pressure as an incentive to the user to comply with the desired behavior.

A participating retailer may provide bonus cash back for compliance by a user in the participating retailer's store or premises using rewards component 126, in an embodiment. For example, a user may get a discount at the retailer location for compliance. A participating financial institution may provide a mechanism to round-up purchases and put the difference in a savings account as a reward to the user for compliance, in an embodiment. For example, if the purchase is for $1.60, an additional $0.40 could be transferred from the user's checking to savings as a reward. A similar transfer could be used as a negative incentive to another user, as funds transferred to a savings account are more difficult to access, in an embodiment. In another embodiment, as a negative incentive, the user may be forced to make a fixed donation to a charity, such as the Humane Society, for every time they violate social distancing requirements. The aforementioned donation could be an escalating amount for repeat offenders, in an embodiment.

The present application provides a rewards points system using rewards component 126, in various embodiments. The user can get points (similar to airline or credit card points) for compliance, and can redeem the points to purchase goods or services at participating merchants, in various embodiments. A user may receive a number of points scaled to the percentage of compliance of social distancing, in various embodiments. In various examples, compliance with one or more rules, regulations, policies, or retailer requirements for social distancing may be detected and enforced using the techniques discussed herein. Satisfaction of one or more conditions associated with a reward may be determined based on the techniques described herein. If the conditions are satisfied, the reward may be granted.

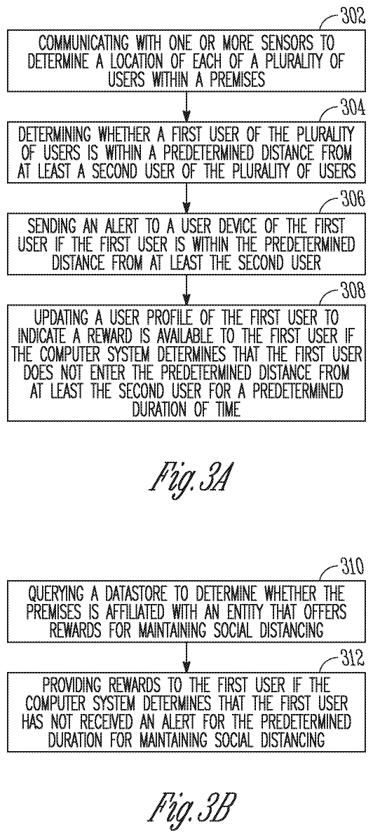

FIG. 3A illustrates an example embodiment of a method for assisting in social distancing compliance, according to various embodiments. The method includes communicating with one or more sensors to determine a location of each of a plurality of users within a premises at step 302, determining whether a user of the plurality of users is within a predetermined distance (e.g., a programmable and adjustable distance) from at least one other user of the plurality of users at step 304, and sending an alert to a user device of the user if the user is within the predetermined distance from the at least one other user of the plurality of users (step 306). At step 308, a user profile of the user is updated to indicate a reward is available to the user if it has been determined that the user has not entered the predetermined distance from another user of the plurality of users for a predetermined duration of time (e.g., a programmable and adjustable duration of time). For instance, the reward may be made available to the user if the user has not received an alert for entering within the predetermined distance from another user for the predetermined duration. As discussed, the reward may be provided by an entity affiliated with the premises to incentivize the user for maintaining social distancing. The method can be combined with the method of FIG. 3B in some embodiments.

FIG. 3B illustrates an example embodiment of a method for enforcing social distancing using technology, according to various embodiments. The method includes querying a datastore to determine whether the premises is affiliated with an entity that offers rewards to users for maintaining social distancing at step 310, and providing a reward or rewards to the user when it has been determined that the user has not entered a predetermined distance from another user of the plurality of users for a predetermined duration of time. For instance, the reward may be made available to the user if the user has not received an alert during the predetermined duration, and the user is present at the premises during the predetermined duration. As discussed, in the described example the premises corresponds to the entity offering rewards to users for maintaining social distancing (step 312). The methods of FIGS. 3A and 3B may be performed using the system components described in FIG. 1, in various embodiments.

FIG. 4 illustrates example embodiments of sensors for use at a premises 450, according to various embodiments. In some embodiments, the proximity detection is done from peer-to-peer (e.g., distance between two users) using Bluetooth, WiFi, or other communication protocols between user devices 410, 412. In some embodiments, the premises owner (such as a retailer or financial institution) can use its own network, such as an existing WiFi network having beacons or repeaters 460, to locate individuals 400, 402 for purposes of the present subject matter. In some embodiments, the premises owner may have a surveil lance system with cameras 470 that is used to locate individuals for purposes of the present subject matter. The surveil lance system may be capable of tagging a person to identify them as they enter the premises, such as by using a motion sensor, camera (if within line of sight) or infrared sensor (triggered by body heat threshold) to detect the person. In various embodiments, the present subject matter determines whether a user 400 of the plurality of users is within a predetermined distance from another user or person 402, and sends an alert to a user device 410 if the user is within the predetermined distance D from the other person.

A method includes communicating with one or more sensors to determine a location of each of a plurality of users within a premises, and determining whether a first user of the plurality of users is within a predetermined distance from one or more of the other users of the plurality of users. An alert is sent to a user device if the user is within the predetermined distance from any of the other users. The method further includes querying a datastore to determine whether the premises is affiliated with an entity that offers (or supplies) rewards to users for maintaining social distancing. A user profile of the user is updated to indicate a reward is available to the user when it has been determined that the user has not entered a predetermined distance from another user of a plurality of users for a predetermined duration of time. For instance, the reward may be made available to the user if the user has not received an alert during the predetermined duration, and the user is present at the premises during the predetermined duration.

According to various embodiments, determining the location of each of the plurality of users within the premises includes using a surveillance system of the premises, using the user device (such as by using a geolocation feature of the device), using near field communications (NFC), using an radio frequency identification (RFID) tag, and/or using communication with a wireless router, beacon and/or camera at the premises. For example, the present system may send a request to the user devices of each of the users to determine the location of the users, and receive a response from the user devices including coordinates of the location of the user devices.

In various embodiments, sending an alert to a user device includes sending one or more of an audible alert, a text alert, or a vibration alert to the user device. Determining whether a user of the plurality of users is within a predetermined distance from other users includes calculating a distance from the user device to one or more personal devices of the other users, in various embodiments. For example, the present system may send a request to the personal devices of each of the users to determine the location of the users, receive a response from the personal devices including coordinates of the location of the personal devices, and compare the received coordinates to determine the distance between personal devices of users. In various embodiments, the predetermined distance is dynamically and/or automatically adjusted based on a layout of the premises. For example, in some stores people in adjacent aisles may be closer than six feet apart, but deemed not in violation of social distancing because of large dividers or high shelves between the aisles. In various embodiments, the application of the present subject matter knows details of the store layout, or uses available video to determine store features to dynamically and/or automatically adjust the predetermined distance. The predetermined duration is programmed to start when the user enters the premises and end when the user leaves the premises, in various embodiments. A sensor at the premises detects the user entering and leaving the premises, or geolocation by the user device can be used to determine the user is entering or leaving the premises in various embodiments.

According to various embodiments, providing rewards to the user when the user has not received the alert for a predetermined duration includes providing points, cash back from a purchase, and/or a discount on a purchase to the user. The user device includes one or more of a smartphone or a wearable device, in various embodiments. In various embodiments, determining whether the premises is affiliated with an entity that offers rewards to users includes using a look-up table of participating entities. For example, the present system may store a list of entities and premises as a look-up table in memory, and the system may query the look-up table to determine whether the premises is affiliated with the entity.

Various embodiments include a system including a computing system comprising one or more processors and a data storage system in communication with the one or more processors, where the data storage system comprises instructions thereon that, when executed by the one or more processors, causes the one or more processors to execute methods of the present subject matter. Various embodiments include a non-transitory computer-readable storage medium, the non-transitory computer-readable storage medium including instructions that, when executed by computers, cause the computers to perform operations to execute methods of the present subject matter.

Various embodiments of the present subject matter generally relate to enforcing social distancing. In one embodiment, the present system determines whether there are users within a location, determines the location of each of the users within the location, determines the proximity of the users to each other, determines if any users are too close to each other, and sends alerts when users are too close to each other, where the alert is sent to the offending users.

Various devices that are able to monitor the location of users are implemented to determine the proximity of one user to a second user. For example, the surveillance system of a retail establishment along with (or independently from) each of the user's phones (e.g., or other user device) may determine whether the first and second users are socially distanced from each other. If the first and second users are not properly spaced apart from each other, i.e., closer than six feet, then an alert will be provided to each of the first user and the second user.

In various embodiments, a retail establishment's surveillance system and/or the user's device determine, in real time, who is social distancing. The determination can be made using near field communications (NFC) of the user's device along with the retail establishment's surveillance system. Moreover, a rewards system can be used to reward users who social distance with others in the retail establishment. In addition to using the surveillance system of a retail establishment, NFC can be used to determine the proximity of users to each other. Furthermore, Wi-Fi systems could be used to determine the location of users where triangulation techniques used via the retail establishment's surveillance system and Wi-Fi that is available at the retail establishment used.

Moreover, when users are socially distancing, awards may be provided to the users. For example, a user may get cash back on their credit card or rewards, i.e., store cash, that are redeemable at the retail establishment. In various embodiments, the provided rewards may include points (cash-back, discount, other standard points options) from participating sponsoring organizations (e.g., retailer who's store you're in, local or national government, etc.) who can determine framework for points allocation based on how strong they want the social distancing incentive. According to various embodiments, the present subject matter may identify who the sponsoring organization is based on geolocation, QR-code scan at entrance to location, or hand-entry of an identifier associated with a location. Other provided rewards may include a sweepstakes entry or a charitable donation, in various embodiments.

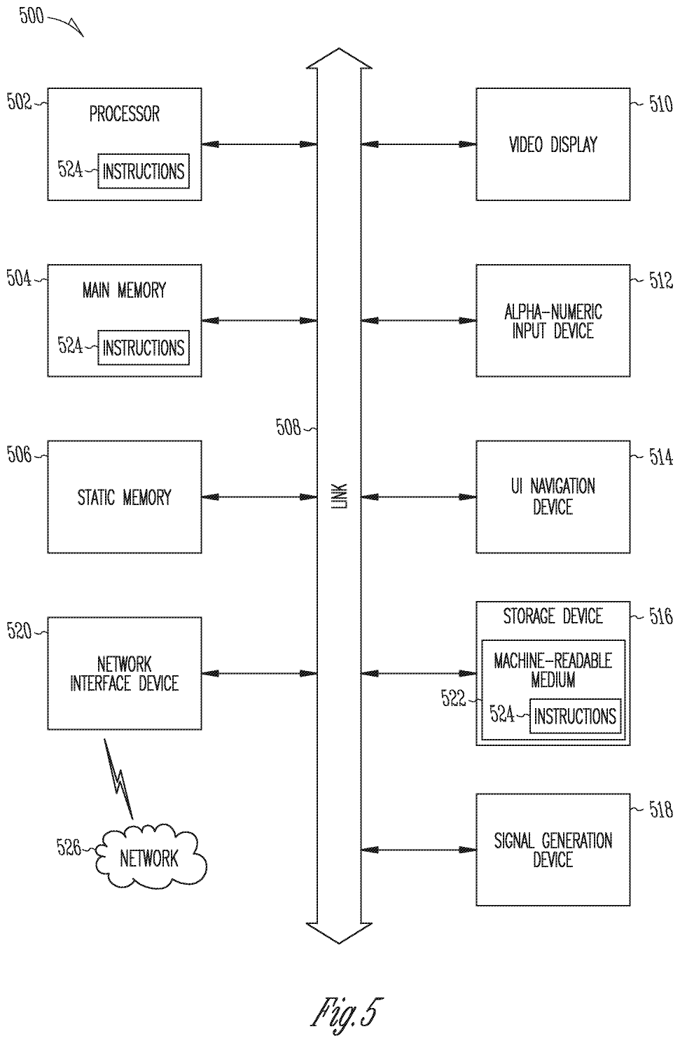

FIG. 5 is a block diagram illustrating a machine in the example form of a computer system 500, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a set-top box (SIB), a personal digital assistant (PDA), a mobile or cellular telephone such as a smartphone, a wearable device such as a smart watch, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

Example computer system 500 includes at least one processor 502 (e.g., a central processing unit (CPU), a graphics processing unit (GPU) or both, processor cores, compute nodes, etc.), a main memory 504 and a static memory 506, which communicate with each other via a link 508 (e.g., bus). The computer system 500 may further include a video display unit 510, an alphanumeric input device 512 (e.g., a keyboard), and a user interface (UI) navigation device 514 (e.g., a mouse). In one embodiment, the video display unit 510, input device 512 and UI navigation device 514 are incorporated into a touch screen display. The computer system 500 may additionally include a storage device 516 (e.g., a drive unit), a signal generation device 518 (e.g., a speaker), a network interface device 520, and one or more sensors (not shown), such as a global positioning system (GPS) sensor, compass, accelerometer, or other sensor.

The data storage device 516 includes a machine-readable medium 522 on which is stored one or more sets of data structures and instructions 524 (e.g., software) embodying or utilized by any one or more of the methodologies or functions described herein. The instructions 524 may include a machine learning system or algorithm, and may also reside, completely or at least partially, within the main memory 504, static memory 506, and/or within the processor 502 during execution thereof by the computer system 500, with the main memory 504, static memory 506, and the processor 502 also constituting machine-readable media.

While the non-transitory computer-readable storage medium 522 is illustrated in an example embodiment to be a single medium, the term “machine-readable medium” or “computer-readable medium” may include a single medium or multiple media (e.g., a centralized or distributed database, and/or associated caches and servers) that store the one or more instructions 524, The term “machine-readable medium” shall also be taken to include any tangible medium that is capable of storing, encoding or carrying instructions (e.g., instructions 524) for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media, Specific examples of machine-readable media include non-volatile memory, including, but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of well-known transfer protocols (e.g., HTTP). Examples of communication networks include a local area network (LAN), a wide area network (WAN), the Internet, mobile telephone networks, plain old telephone system (POTS) networks, and wireless data networks (e.g., Wi-Fi, 3G, and 6G LTE/LTE-A or WiMAX networks). The term “transmission medium” shall be taken to include any intangible medium that is capable of storing, encoding; or carrying instructions for execution by the machine, and includes digital or analog communications signals or other intangible medium to facilitate communication of such software.

The above description is intended to be illustrative, and not restrictive. For example, the above-described examples (or one or more aspects thereof) may be used in combination with others. Other embodiments may be used, such as by one of ordinary skill in the art upon reviewing the above description. The Abstract is to allow the reader to quickly ascertain the nature of the technical disclosure, for example, to comply with 37 C.F.R. § 1.72(b) in the United States of America. It is submitted with the understanding that it will not be used to interpret or limit the scope or meaning of the claims.

Also, in the above Detailed Description, various features may be grouped together to streamline the disclosure. However, the claims may not set forth every feature disclosed herein as embodiments may feature a subset of said features. Further, embodiments may include fewer features than those disclosed in a particular example. Thus, the following claims are hereby incorporated into the Detailed Description, with a claim standing on its own as a separate embodiment. The scope of the embodiments disclosed herein is to be determined with reference to the appended claims, along with the full scope of equivalents to which such claims are entitled.

1. A computer-implemented method comprising:
communicating, by a computer system, with one or more sensors to determine a location of each of a plurality of users within a premises;
determining, by the computer system, whether a first user of the plurality of users has elected to participate in a rewards program;
determining, by the computer system, whether the first user of the plurality of users is within a predetermined distance from at least a second user of the plurality of users;
sending, by the computer system, an alert to a user device of the first user if the first user is within the predetermined distance from at least the second user;
determining, by the computer system, whether the first user has responded to the alert by moving to or beyond the predetermined distance from at least the second user;
determining, by the computer system, whether the premises is affiliated with an entity that offers rewards for maintaining social distancing, including using a geographic location of the premises as an input to query a datastore; and
updating, by the computer system, a user profile of the first user to provide a first reward to the first user for electing to participate in the rewards program using a first component of the rewards program and to provide a second reward to the first user when the computer system determines that the first user has moved to or beyond the predetermined distance from at least the second user for a predetermined duration of time using a second component of the rewards program, the second reward being offered to the first user by the entity for maintaining social distancing.

2. The computer-implemented method of claim 1, wherein communicating with one or more sensors to determine the location of each of the plurality of users within the premises includes transmitting an interrogation signal to a surveillance system of the premises and receiving a response from the surveillance system.

3. The computer-implemented method of claim 1, wherein communicating with one or more sensors to determine the location of each of the plurality of users within the premises includes transmitting an interrogation signal to the first user device and receiving a response from the first user device.

4. The computer-implemented method of claim 1, wherein communicating with one or more sensors to determine the location of each of the plurality of users within the premises includes transmitting an interrogation signal to a wireless router at the premises and receiving a response from the wireless router.

5. The computer-implemented method of claim 1, wherein sending the alert to the first user device includes sending one or more of an audible alert, a text alert, or a vibration alert to the first user device.

6. The computer-implemented method of claim 1, wherein determining whether the first user of the plurality of users is within the predetermined distance from at least the second user of the plurality of users includes calculating a distance from the first user device to a second user device of the second user.

7. The computer implemented method of claim 6, further comprising determining, by the computer system, whether the first user is within the predetermined distance from a third user of the plurality of users,
wherein determining whether the first user of the plurality of users is within the predetermined distance from the third user of the plurality of users includes calculating a distance from the first user device to a third user device of the third user.

8. The computer-implemented method of claim 1, wherein communicating with one or more sensors to determine the location of each of a plurality of users within the premises includes transmitting an interrogation signal to a radio frequency identification (RFID) tag and receiving a response from the RFID tag.

9. The computer-implemented method of claim 1, wherein the predetermined distance is dynamically adjusted based on a layout of the premises.

10. The computer-implemented method of claim 1, wherein the predetermined duration is programmed to start when the first user enters the premises and end when the first user leaves the premises.

11. A system comprising:
a computer system comprising one or more processors and a data storage system in communication with the one or more processors, wherein the data storage system comprises instructions thereon that, when executed by the one or more processors, causes the one or more processors to:
communicate with one or more sensors to determine a location of each of a plurality of users within a premises;
determine whether a first user of the plurality of users has elected to participate in a rewards program;
determine whether the first user of the plurality of users is within a predetermined distance from at least a second user of the plurality of users;
send an alert to a user device of the first user if the first user is within the predetermined distance from at least the second user;
determine whether the first user has responded to the alert by moving to or beyond the predetermined distance from at least the second user;
determine whether the premises is affiliated with an entity that offers rewards for maintaining social distancing, including using a geographic location of the premises as an input to query a datastore; and
update a user profile of the first user to provide a first reward to the first user for electing to participate in the rewards program using a first component of the rewards program and to provide a second reward to the first user when the computer system determines that the first user has moved to or beyond the predetermined distance from at least the second user for a predetermined duration of time using a second component of the rewards program, the second reward being offered to the first user by the entity for maintaining social distancing.

12. The system of claim 11, comprising updating the user profile to provide points to the first user when the computer system determines that the first user has not received the alert for the predetermined duration.

13. The system of claim 11, comprising updating the user profile to provide cash back from a purchase to the first user when the computer system determines that the first user has not received the alert for the predetermined duration.

14. The system of claim 11, comprising updating the user profile to provide a discount on a purchase to the first user when the computer system determines that the first user has not received the alert for the predetermined duration.

15. The system of claim 11, wherein the predetermined duration is configured to start when a sensor at the premises detects the first user entering the premises.

16. The system of claim 11, wherein the user device includes one or more of a smartphone or a wearable device.

17. A non-transitory computer-readable storage medium, the non-transitory computer-readable storage medium including instructions that, when executed by computers, cause the computers to perform operations of:
communicating with one or more sensors to determine a location of each of a plurality of users within a premises;
determining whether a first user of the plurality of users has elected to participate in a rewards program,
determining whether the first user of the plurality of users is within a predetermined distance from at least a second user of the plurality of users;
sending an alert to a user device of the first user if the first user is within the predetermined distance from at least the second user;
determining whether the first user has responded to the alert by moving to or beyond the predetermined distance from at least the second user;
determining whether the premises is affiliated with an entity that offers rewards for maintaining social distancing, including using a geographic location of the premises as an input to query a datastore; and
updating a user profile of the first user to provide a first reward to the first user for electing to participate in the rewards program using a first component of the rewards program and to provide a second reward to the first user when the computers determine that the first user has moved to or beyond the predetermined distance from at least the second user for a predetermined duration of time using a second component of the rewards program, the second reward being offered to the first user by the entity for maintaining social distancing.

18. The non-transitory computer-readable storage medium of claim 17, wherein communicating with one or more sensors to determine the location of each of the plurality of users includes communicating with a geolocation feature of the user device.

19. The non-transitory computer-readable storage medium of claim 17, wherein communicating with one or more sensors to determine the location of each of the plurality of users includes communicating with a beacon at the premises.

20. The non-transitory computer-readable storage medium of claim 17, wherein querying the datastore to determine whether the premises is affiliated with the entity that offers rewards includes using a look-up table of participating entities.
